(19) United States
(12) Patent Application Publication
(10) Pub. No.: US 2023/0127531 A1
(43) Pub. Date: Apr. 27, 2023

(54) FILTER

(71) Applicant: LG ELECTRONICS INC., Seoul (KR)

(72) Inventors: Sunyoung MOON, Seoul (KR); Hyungho PARK, Seoul (KR)

(21) Appl. No.: 17/915,223

(22) PCT Filed: Jan. 19, 2021
(86) PCT No.: PCT/KR2021/000723

§ 371 Date: Sep. 28, 2022

(30) Foreign Application Priority Data
Mar. 31, 2020 (KR) 10-2020-0039209

(51) Int. Cl. A01N 59/16; B01J 20/02; B01J 20/06; B01J 20/26; A61L 9/014; A01N 59/20; A01N 25/10; B01D 39/16; B01D 46/00

(52) U.S. Cl. CPC A01N 59/16; B01J 20/0233; B01J 20/0225; B01J 20/06; B01J 20/262; A61L 9/014; A01N 59/20; A01N 25/10; B01D 39/16; B01D 46/0028; A61L 2209/14; B01D 2239/0622; B01D 2239/0442; B01D 2279/65

(57) ABSTRACT

A filter is disclosed. The filter of the present disclosure includes: a support layer; and a filter layer coupled to the support layer, wherein the filter layer is formed by a melt blown method with, as a melt, a thermoplastic resin-including first base and a first antibacterial agent, wherein the first antibacterial agent includes an antibacterial metal or antibacterial metal oxide.

### TECHNICAL FIELD

The present disclosure relates to a filter. Particularly, the present disclosure relates to a filter manufactured based on antibacterial fibers and applicable to an air purifier.

### BACKGROUND ART

An air purifier is a device that improves indoor air quality by purifying indoor polluted air. In general, the air purifier includes a filter that filters out substances of a fine size such as fine dust, bacteria, or viruses floating in the air.

In recent years, antibacterial agents have been added to filters by dyeing, coating, etc. to inhibit the growth or survival of microorganisms in the air passing through the filters.

However, there were concerns about the decrease in antibacterial performance due to the loss of the antibacterial agent resulting from filter washing, filter wear, etc. in the existing methods of adding the antibacterial agents such as dyeing, coating, etc.

### DISCLOSURE OF INVENTION

### Technical Problem

An object of the present disclosure is to solve the above-mentioned problems and other problems.

Another object may be to provide a filter capable of maintaining antibacterial performance above a certain level by preventing the loss of an antibacterial agent due to filter washing, filter wear, etc.

Another object may be to provide a filter capable of preventing the discharge of chemicals harmful to a human body from the filter.

Another object may be to provide a filter manufactured by a melt blown method and capable of easily improving the antibacterial performance by adjusting the content of the antibacterial agent.

Another object may be to provide a filter capable of improving dust collection performance along with the antibacterial performance.

### Solution to Problem

According to an aspect of the present disclosure for achieving the aforementioned purposes, there is provided a filter including a support layer and a filter layer coupled to the support layer, wherein the filter layer is formed by the melt blown method with, as a melt, a thermoplastic resin-including first base and a first antibacterial agent, and the first antibacterial agent includes an antibacterial metal or antibacterial metal oxide.

According to another aspect of the present disclosure, the support layer may be formed by the melt blown method with, as a melt, a thermoplastic resin-including second base and a second antibacterial agent, the second antibacterial agent may include an antibacterial metal or antibacterial metal oxide.

According to another aspect of the present disclosure, the first antibacterial agent and the second antibacterial agent may be the same as each other.

According to another aspect of the present disclosure, the first antibacterial agent and the second antibacterial agent may be different from each other.

According to another aspect of the present disclosure, the content of the first antibacterial agent for the filter layer may be 0.1 to 5%.

According to another aspect of the present disclosure, a melt index of the melt may be, under certain conditions, 400 to 900 based on a melt index of 900 to 1,200 of a comparative melt including only the first base.

According to another aspect of the present disclosure, the support layer the support layer 332 may include polypropylene (PP), polyethylene terephthalate (PET), polytetrafluoroethylene (PTFE), staple fiber, or acrylic.

According to another aspect of the present disclosure, the first base may include polyethylene terephthalate (PET), polypropylene (PP), or polytetrafluoroethylene (PTFE).

According to another aspect of the present disclosure, the metal may include silver (Ag), gold (Au), or platinum (Pt), and the metal oxide may include zinc oxide (ZnO), copper oxide (Cu₂O, CuO), or titanium dioxide (TiO₂).

According to another aspect of the present disclosure, the first antibacterial agent may be formed in the form of a master batch.

### Advantageous Effects of Invention

The effect of the filter according to the present disclosure will be described as follows.

According to at least one of the embodiments of the present disclosure, there can be provided the filter capable of maintaining the antibacterial performance above a certain level by preventing the loss of the antibacterial agent due to filter washing, filter wear, etc.

According to at least one of the embodiments of the present disclosure, there can be provided the filter capable of preventing the discharge of chemicals harmful to a human body from the filter.

According to at least one of the embodiments of the present disclosure, there can be provided the filter manufactured by the melt blown method and capable of easily improving the antibacterial performance by adjusting the content of the antibacterial agent.

Further, the scope of applicability of the present disclosure will be clearly understood with reference to the following detailed description. However, since various changes and modifications within the spirit and scope of the present disclosure can be clearly understood by a person having ordinary skill in the art, it should be understood that the detailed description and specific embodiments such as preferred embodiments of the present disclosure are given by way of example only.

### BRIEF DESCRIPTION OF DRAWINGS

FIG. 1 is a front view of an air purifier to which a filter according to an embodiment of the present disclosure is applied.

FIG. 2 is a cross-sectional view of the air purifier shown in FIG. 1.

FIG. 3 is a perspective view of a filter assembly including the filter according to an embodiment of the present disclosure.

FIG. 4 is an exploded perspective view of the filter assembly shown in FIG. 3.

FIG. 5 is a perspective view of a part cut away from the filter assembly shown in FIG. 3.

FIG. 6 is a view illustrating a melt blown method, which is a method of manufacturing the filter according to an embodiment of the present disclosure.

FIGS. 7A to 7D are views showing the content of an antibacterial agent in fibers of the filter according to an embodiment of the present disclosure.

FIGS. 8 and 9 are tables showing the correlation between the content of the antibacterial agent and antibacterial performance depending on a melt index in the melt blown method, which is the method of manufacturing the filter according to an embodiment of the present disclosure.

FIG. 10 is a table showing the emission amount of zinc oxide in the method of manufacturing the filter according to an embodiment of the present disclosure.

### MODE FOR CARRYING OUT INVENTION

Hereinafter, with reference to the appended drawings, the embodiments disclosed in the present specification will be described in detail, but, regardless of a drawing reference number, the same or similar components will have the same reference number without a repetition of the description thereof.

The terms “module” and “unit/part” for components used in the following description are used interchangeably only in consideration of convenience of writing the specification, so they themselves do not have distinct meanings or roles.

In addition, when it is determined that a detailed description of a related art involved in describing an embodiment disclosed in the specification may obscure the gist of the embodiment, the detailed description will not be provided. Furthermore, the appended drawings are only for easy understanding of the embodiments disclosed in the specification, and the technology disclosed in the specification is not limited by the drawings and should be deemed to include all modifications, equivalents, and substitutes included in the technology and scope of the present disclosure.

Terms including ordinal numbers such as “first,” “second,” etc. may be used to describe various components, but the components are not limited by the terms. The terms are used only for the purpose of distinguishing one component from another.

When a certain component is described to be “connected” or “coupled” to another component, it should be understood that the component may be directly connected or coupled to the other component or another component may exist therebetween. On the other hand, when a certain component is described to be “directly connected” or “directly coupled” to another component, it should be understood that no other component exists therebetween.

Expressions in the singular form include the meaning of the plural form unless they clearly mean otherwise in the context.

In the following description, even when an embodiment is described with reference to a specific drawing, a reference number not shown in the drawing may be used, if necessary, on the condition that it is shown in the other drawings.

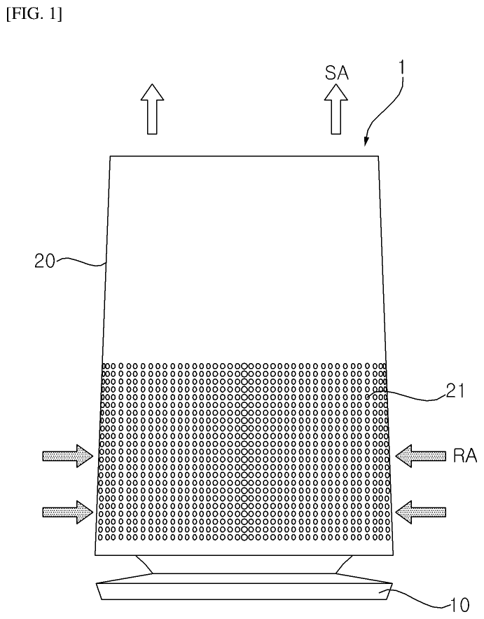

Referring to FIG. 1, an air purifier 1 may include a base 10 and a case 20. Here, the air purifier 1 may be referred to as an air conditioner.

For example, the base 10 may be formed in the shape of a circular plate as a whole and may be capable of supporting the rest of the components of the air purifier 1. For example, the case 20 may be formed in the shape of a truncated cone as a whole.

A suction hole 21 may be formed in a portion of a side surface of the case 20 to provide room air RA into the interior of the case 20. For example, the suction hole 21 may be formed along the circumference of the case 20 contiguous to a lower end of the case 20. In this case, the room air RA may flow in a horizontal direction and may be introduced into the case 20.

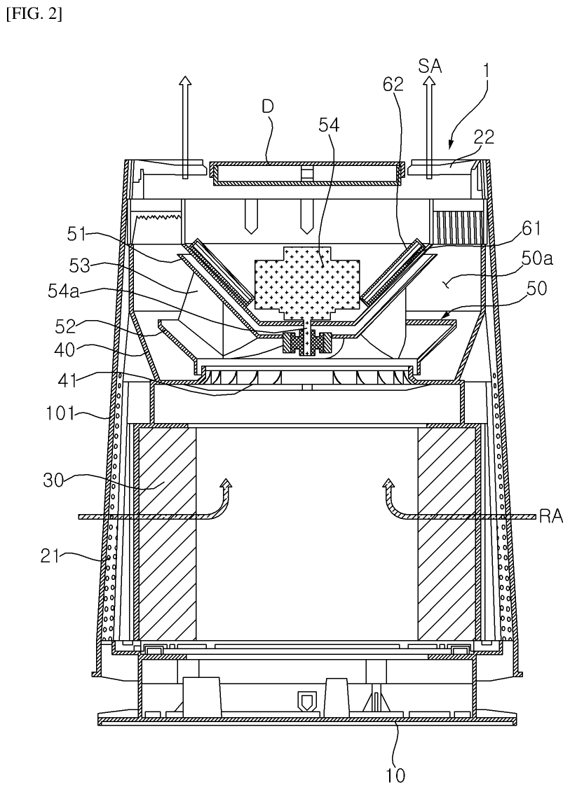

A discharge hole 22 (see FIG. 2) may be formed in a portion of an upper surface of the case 20 to pass through the air purifier 1 and may be capable of providing purified supplying air SA to the room. In this case, the supplying air SA may flow in a vertical direction and may be discharged to the outside of the case 20.

Referring to FIG. 2, the air purifier 1 may include a filter assembly 30 and a fan module 50. The filter assembly 30 may be installed inside the case 20 contiguous to the suction hole 21 and will be described in more detail below.

The fan module 50 may be installed inside the case 20 and positioned above the filter assembly 30. The fan module 50 may be installed in a fan housing 40 fixed to the inside of the case 20. The fan module 50 may cause a flow of air from the suction hole 21 to the discharge hole 22. In this case, air may be introduced into the fan module 50 through an inlet part 41 of the fan housing 40.

Specifically, the fan module 50 may include a hub 51, a shroud 52, a blade 53, and a rotation motor 54. In this case, the hub 51 may be coupled to a rotation shaft 54a of the rotation motor 54, and the shroud 52 may be spaced apart from the hub 51. In addition, a plurality of blades 53 may be provided, positioned between the hub 51 and the shroud 52, and rotated by the power of the rotation motor 54 so that air flow may be caused.

That is, based on the operation of the rotation motor 54, after the room air RA introduced through the suction hole 21 may be purified while passing through the filter assembly 30, it may be supplied into the room as the supplying air SA through the fan module 50 and then through the discharge hole 22.

A sound absorbing material 61 may be installed at a mount 62 fixed to the inside of the case 20 and located above an air flow path 50a passing through the fan module 50. For example, the sound absorbing material 61 may be made of a porous member including a material such as resin, rubber, sponge, or polyurethane foam. In this case, flow noise that air passing through the air flow path 50a causes may be reduced by the sound absorbing material 61.

A display unit D may be installed on an upper portion of the air purifier 1. For example, the display unit D may display operation information of the air purifier 1.

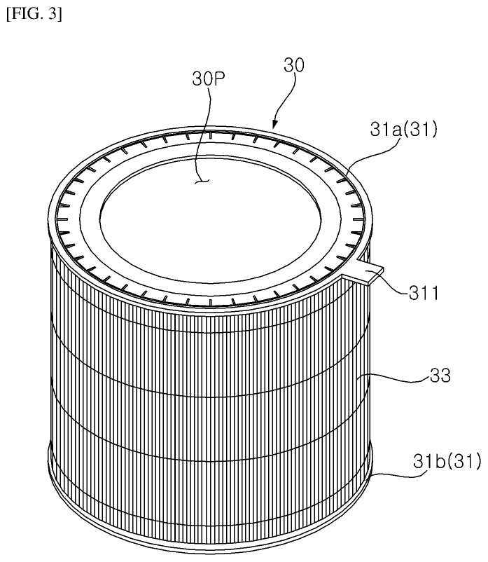

Referring to FIG. 3, the filter assembly 30 may be formed in the shape of a cylinder as a whole and may include an opening 30P therein. Here, the opening 30P may be formed along a vertical direction. In this case, based on the operation of the rotation motor 54, the room air RA introduced through the suction hole 21 (see FIG. 2) may be purified while flowing from the outer circumferential surface to the inner circumferential surface of the filter assembly 30 and may then flow upward through the opening 30P.

A frame 31 may be provided at upper and lower ends of the filter assembly 30 to function as a support so that the filter assembly 30 may maintain the shape of a cylinder. The frame 31 may include a first upper frame 31a provided at the upper end of the filter assembly 30 and a first lower frame 31b provided at the lower end of the filter assembly 30.

Meanwhile, a strap 311 may be provided on one side of the first upper frame 31a. In this case, it may be possible that a user grabs the strap 311 and pulls it in a horizontal direction to take out the filter assembly 30 from the case 20 in a horizontal direction. Here, the strap 311 may be referred to as a handle.

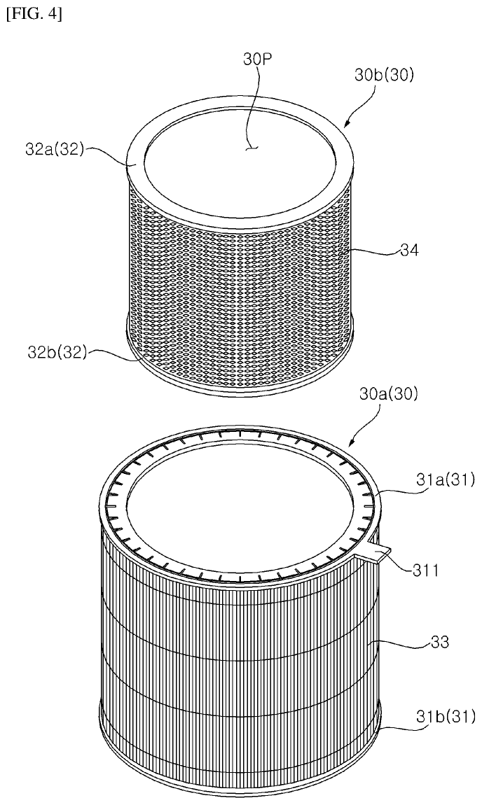

Referring to FIG. 4, the filter assembly 30 may include a first assembly 30a and a second assembly 30b. The first assembly 30a may form the exterior of the filter assembly 30, and the second assembly 30b may be inserted into the first assembly 30a. In this case, the opening 30P may be formed inside the second assembly 30b.

The first upper frame 31a and the first lower frame 31b may be provided at upper and lower ends of the first assembly 30a, respectively. In this case, the first upper frame 31a and the first lower frame 31b may be formed in the shape of a ring as a whole.

A first filter 33 may be coupled to the first upper frame 31a and the first lower frame 31b therebetween. As a result, the first filter 33 may be supported by the first upper frame 31a and the first lower frame 31b to maintain the shape of a cylinder.

A second upper frame 32a and a second lower frame 32b may be provided at upper and lower ends of the second assembly 30b, respectively. In this case, the second upper frame 32a and the second lower frame 32b may be formed in the shape of a ring as a whole. In addition, the outer circumferential surface of the second upper frame 32a may face the inner circumferential surface of the first upper frame 31a, and the outer circumferential surface of the second lower frame 32b may face the inner circumferential surface of the first lower frame 31b.

A second filter 34 may be coupled to the second upper frame 32a and the second lower frame 32b therebetween. As a result, the second filter 34 may be supported by the second upper frame 32a and the second lower frame 32b to maintain the shape of a cylinder.

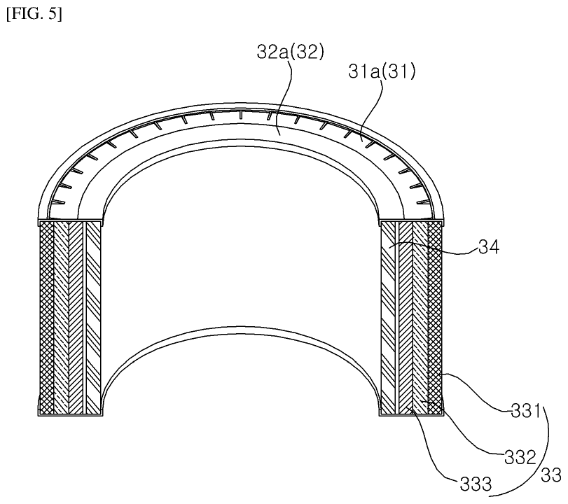

Referring to FIG. 5, the first filter 33 may include a pre-filter 331 and a HEPA filter 332 and 333.

The pre-filter 331 may be located at the outermost portion of the first filter 33. The pre-filter 331 may filter out animal hair, lint, hair, large dust, etc. For example, the pre-filter 331 may have a mesh structure so that a plurality of through holes may be formed therein. Meanwhile, the pre-filter 331 may be detachably provided in the first filter 33 to be washed and reused as needed. For example, one end and the other end of the pre-filter 331 in the circumferential direction of the first filter 33 may be coupled in a velcro manner.

The HEPA filter 332 and 333 may be located inside the pre-filter 331. Here, “HEPA” is an abbreviation for High Efficiency Particulate Air. The HEPA filter 332 and 333 may filter out microscopic substances such as fine dust, bacteria, or viruses. For example, the HEPA filter 332 and 333 may have the mesh structure so that a plurality of through holes may be formed therein. In particular, an antibacterial agent may be added to the HEPA filter 332 and 333 to inhibit the growth or survival of microorganisms in the air passing through the filter, which will be described in more detail below.

The second filter 34 may be a deodorizing filter 34. The deodorizing filter 34 may be located inside the HEPA filter 332 and 333. For example, a plurality of through holes may be formed in the deodorizing filter 34. For example, the deodorizing filter 34 may be an activated carbon filter or a carbon filter and may remove odors and/or harmful gases contained in the air by a chemical adsorption method. Furthermore, the deodorizing filter 34 may be coated with a photocatalyst activated by light. In this case, the deodorizing filter 34 may remove odors by decomposing harmful substances contained in the air through a photochemical reaction.

Accordingly, based on the operation of the rotation motor 54, the room air RA introduced through the suction hole 21 (see FIG. 2) may be purified while sequentially passing through the pre-filter 331, the HEPA filter 332 and 333, and the deodorizing filter 34.

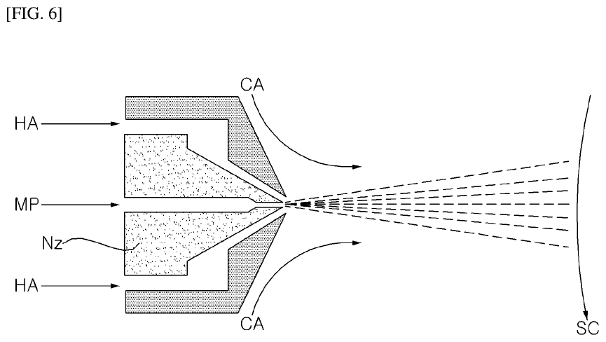

Referring to FIGS. 5 and 6, the HEPA filter 332 and 333 may include a support layer 332 and a filter layer 333. Meanwhile, although FIG. 5 shows that the support layer 332 is located outside the filter layer 333, it may also be possible that the support layer 332 is located inside the filter layer 333.

The support layer 332 may be coupled to the first upper frame 31a and the first lower frame 31b therebetween. As a result, the support layer 332 may be supported by the first upper frame 31a and the first lower frame 31b. For example, the support layer 332 may include polypropylene (PP), polyethylene terephthalate (PET), polytetrafluoroethylene (PTFE), staple fiber, or acrylic.

The filter layer 333 may be coupled to the support layer 332 between the first upper frame 31a and the first lower frame 31b. Accordingly, the filter layer 333 may be supported by the support layer 332. For example, the filter layer 333 may be coupled to the support layer 332 by a hot-melt adhesive. In this case, the thickness of the support layer 332 may be thicker than the thickness of the filter layer 333.

The support layer 332 and the filter layer 333 may have a corrugated shape in which ridges and grooves extending long in a vertical direction as a whole are alternately formed. That is, the support layer 332 and the filter layer 333 may be formed in the corrugated shape along the circumferential direction of the first filter 33. As a result, the contact area with the air passing through the support layer 332 and the filter layer 333 may be increased so that the air purification performance of the HEPA filter 332 and 333 may be improved.

The filter layer 333 may be formed while the composition thereof is melt-spinning. For example, the filter layer 333 may be manufactured by a melt blown method. Specifically, a molten polymer MP may be spun through a nozzle Nz, and high-temperature and high-speed air HA supplied toward the end of the nozzle Nz may blow the molten polymer MP spun from the nozzle Nz to form microfibers. The microfibers manufactured in such a manner may be laminated or wound on a screen SC rotated by a motor (not shown).

The support layer 332 may also be manufactured by the melt-spinning or the melt blown method as is the filter layer 333.

As described above, since the HEPA filter 332 and 333, which is manufactured by the melt blown method to have an antibacterial function, may have an antibacterial agent impregnated in its fibers, it may be capable of maintaining the antibacterial performance at a certain level or more by preventing the loss of the antibacterial agent due to moisture contained in the air or its wear.

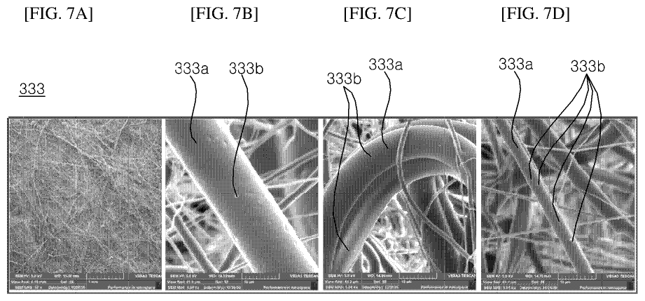

Referring to FIGS. 6 and 7, the filter layer 333 may include a base 333a and an antibacterial agent 333b. That is, to produce the filter layer 333, the base 333a and the antibacterial agent 333b may be transferred to the nozzle Nz by an extruder (not shown) in a molten state and spun from the nozzle Nz toward the screen SC. Here, the base 333a may be referred to as a yarn or fiber yarn before the melt-spinning and may be referred to as a fiber or microfiber after the melt-spinning. In addition, the antibacterial agent 333b may be referred to as an additive.

The base 333a may include a thermoplastic resin. For example, the base 333a may include polyethylene terephthalate (PET), polypropylene (PP), or polytetrafluoroethylene (PTFE). The base 333a may be a chip before melting.

The antibacterial agent 333b may include an antibacterial metal or an antibacterial metal oxide. In this case, since the metallic antibacterial agent 333b may have a (−) charge, the performance of collecting dust usually having a (+) charge to the HEPA filter 332 and 333 may be improved. For example, the antibacterial metal may include silver (Ag), gold (Au), or platinum (Pt), and the antibacterial metal oxide may include zinc oxide (ZnO), copper oxide (Cu₂O, CuO), or titanium dioxide (TiO₂). The antibacterial agent 333b may be formed in the form of a powder or a master batch before melting.

Accordingly, the content of the antibacterial agent 333b in the filter layer 333 manufactured by the melt blown method may be adjusted based on the mixing ratio of the antibacterial agent 333b to the base 333a. The content of the antibacterial agent 333b may be calculated based on the ratio of fibers 333a of the filter layer 333 and the antibacterial agent 333b impregnated therein that are measured or observed through a scanning electron microscope (SEM) (see FIG. 7).

That is, when the antibacterial agent 333b is provided in “a” part(s) by weight with respect to 100 parts by weight of the base 333a as the melt of the filter layer 333 (see figure (a) of FIG. 7) produced in the form of microfibers by the melt blown method, the content of the antibacterial agent 333b in the filter layer 333 may be 1% (see figure (b) of FIG. 7). In addition, when the antibacterial agent 333b is provided in “b” part(s) by weight with respect to 100 parts by weight of the base 333a as the melt of the filter layer 333, the content of the antibacterial agent 333b in the filter layer 333 may be 3% (see figure (c) of FIG. 7). Furthermore, when the antibacterial agent 333b is provided in “c” part(s) by weight with respect to 100 parts by weight of the base 333a as the melt of the filter layer 333, the content of the antibacterial agent 333b in the filter layer 333 may be 5% (see figure (d) of FIG. 7). Here, “b” may be greater than “a,” and “c” may be greater than “b.”

In this case, the antibacterial function of the filter may not be performed when the content of the antibacterial agent 333b is excessively low, and the mass production rate of the filter may be reduced when the content of the antibacterial agent 333b is excessively high. In other words, the content of the antibacterial agent 333b may be preferably 0.1 to 5%.

In the meantime, the support layer 332 may also be manufactured by the melt blown method and have the antibacterial function as is the filter layer 333. In this case, both the support layer 332 and the filter layer 333 may include the antibacterial metal or the antibacterial metal oxide targeting the same target bacteria or microorganism. Alternatively, the support layer 332 and the filter layer 333 may include the antibacterial metal or the antibacterial metal oxide targeting different target bacteria or microorganisms.

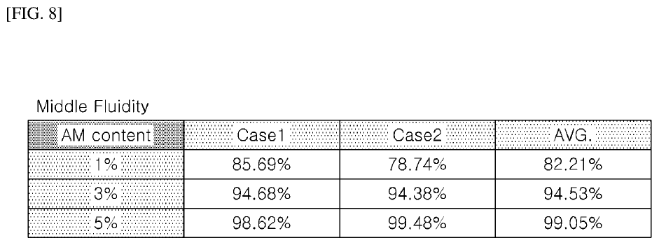

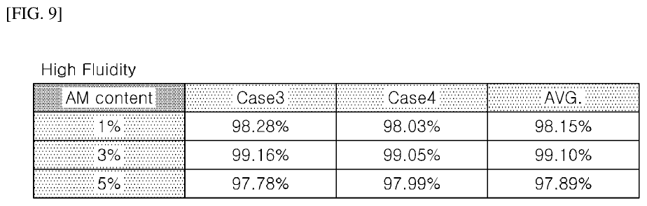

Referring to FIGS. 8 and 9, depending on the melt index (MI), the correlation between the content of the antibacterial agent (AM content) and the antibacterial performance may be different. The melt index (MI) is an index indicating the flow rate of a melt extruded from a piston under certain conditions, e.g., temperature conditions, and the level of the fluidity of the melt. For example, the unit of the melt index (MI) may be g/10 min. Here, the melt index (MI) may be referred to as a melt flow index.

Specifically, the melt index (MI) may be 900 to 1,200 when the filter layer 333 is manufactured by the melt blown method based on only the base 333a under certain conditions. In the case that the melt index (MI) is 400 to 900 and 900 to 1,500 when the filter layer 333 is, under the same conditions, manufactured by the melt blown method by mixing the antibacterial agent 333b in the form of the master batch with the base 333a, it may correspond to middle fluidity and high fluidity, respectively. Here, it can be understood that the high fluidity means better fluidity of a melt compared to the middle fluidity.

In addition, an evaluation of the antibacterial performance was made based on ISO 20743 based on Staphylococcus aureus. That is, the evaluation of the antibacterial performance was performed by measuring the reduction or removal rate of Staphylococcus aureus exposed to the filter layer 333 to which antibacterial treatment had been applied for 18 hours as compared to a filter without the antibacterial treatment.

FIG. 8 shows the antibacterial performance based on the content of the antibacterial agent (AM content) in the case of the middle fluidity. That is, Staphylococcus aureus exposed to the filter layer 333 having the content of the antibacterial agent of 1% for 18 hours was reduced by 85.69% (Case 1) or 78.74% (Case 2), indicating an antibacterial performance of 82.21% on average (AVG). In addition, Staphylococcus aureus exposed to the filter layer 333 having the content of the antibacterial agent of 3% for 18 hours was reduced by 94.68% (Case 1) or 94.38% (Case 2), indicating an antibacterial performance of 94.53% on average (AVG). Furthermore, Staphylococcus aureus exposed to the filter layer 333 having the content of the antibacterial agent of 5% for 18 hours was reduced by 98.62% (Case 1) or 99.48% (Case 2), indicating an antibacterial performance of 99.05% on average (AVG).

FIG. 9 shows the antibacterial performance based on the content of the antibacterial agent (AM content) in the case of the high fluidity. That is, Staphylococcus aureus exposed to the filter layer 333 having the content of the antibacterial agent of 1% for 18 hours was reduced by 98.28% (Case 3) or 98.03% (Case 4), indicating an antibacterial performance of 98.15% on average (AVG). In addition, Staphylococcus aureus exposed to the filter layer 333 having the content of the antibacterial agent of 3% for 18 hours was reduced by 99.16% (Case 3) or 99.05% (Case 4), indicating an antibacterial performance of 99.10% on average (AVG). Furthermore, Staphylococcus aureus exposed to the filter layer 333 having the content of the antibacterial agent of 5% for 18 hours was reduced by 97.78% (Case 3) or 97.99% (Case 4), indicating an antibacterial performance of 97.89% on average (AVG).

As such, it is seen that the antibacterial performance clearly tends to increase as the content of the antibacterial agent (AM content) of the filter layer 333 increases in the case of the middle fluidity, compared to the high fluidity. In other words, it is in the case of the middle fluidity that it is easy to achieve a desired antibacterial performance by adjusting the content of the antibacterial agent in the filter layer 333.

What has been described above may be equally applied to the support layer 332 manufactured by the melt blown method and having the antibacterial function.

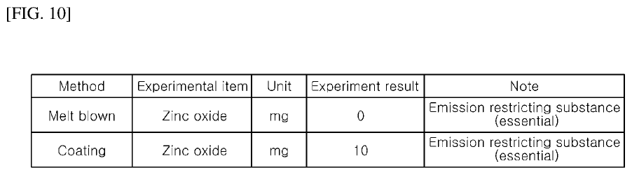

Referring to FIG. 10, when the filter layer 333 having the antibacterial function is manufactured by the melt blown method, it may be possible to prevent emission of an emission restricting substance such as zinc oxide.

Specifically, in the case of a filter layer prepared by a coating method (i.e., a method in which an antibacterial agent is coated on fibers) at a temperature of 23.1° C. and a relative humidity of 46% RH and having antibacterial function, 10 mg of zinc oxide may be released, whereas, in the case of the filter layer 333 prepared by the melt blown method (i.e., a method in which an antibacterial agent is impregnated into fibers by spinning a melt of the antibacterial agent and yarn) and having the antibacterial function, no zinc oxide may be released.

Therefore, it may be desirable to prepare the filter layer 333 having the antibacterial function by the melt blown method in resolving a user's chemophobia.

What has been described above may be equally applied to the support layer 332 manufactured by the melt blown method and having the antibacterial function.

Certain embodiments and other embodiments of the present disclosure described above are not mutually exclusive or distinct. The features or functions of the certain embodiments and other embodiments of the present disclosure described above may be used or combined with one another.

For example, it means that feature A described in a specific embodiment and/or drawing may be combined with feature B described in another embodiment and/or drawing. That is, even if a combination of features is not directly described, it means that the combination of features is possible unless described otherwise.

The detailed description above should not be construed as restrictive in all respects and should be deemed exemplary. The scope of the present disclosure should be determined by a reasonable interpretation of the appended claims, and all modifications within the equivalent scope of the present disclosure are included in the scope of the present disclosure.

1. A filter comprising:
a support layer; and
a filter layer coupled to the support layer,
wherein the filter layer is formed by a melt blown method with, as a melt, a thermoplastic resin-including first base and a first antibacterial agent,
wherein the first antibacterial agent includes an antibacterial metal or antibacterial metal oxide.

2. The filter of claim 1, wherein the support layer is formed by the melt blown method with, as a melt, a thermoplastic resin-including second base and a second antibacterial agent,
wherein the second antibacterial agent includes an antibacterial metal or antibacterial metal oxide.

3. The filter of claim 2, wherein the first antibacterial agent and the second antibacterial agent are the same as each other.

4. The filter of claim 2, wherein the first antibacterial agent and the second antibacterial agent are different from each other.

5. The filter of claim 1, wherein the content of the first antibacterial agent for the filter layer is 0.1 to 5%.

6. The filter of claim 5, wherein a melt index of the melt is, under certain conditions, 400 to 900 based on a melt index of 900 to 1,200 of a comparative melt including only the first base.

7. The filter of claim 1, wherein the support layer the support layer 332 includes polypropylene (PP), polyethylene terephthalate (PET), polytetrafluoroethylene (PTFE), staple fiber, or acrylic.

8. The filter of claim 1, wherein the first base includes polyethylene terephthalate (PET), polypropylene (PP), or polytetrafluoroethylene (PTFE).

9. The filter of claim 1, wherein the metal includes silver (Ag), gold (Au), or platinum (Pt), and the metal oxide includes zinc oxide (ZnO), copper oxide (Cu₂O, CuO), or titanium dioxide (TiO₂).

10. The filter of claim 1, wherein the first antibacterial agent is formed in the form of a master batch.
